SEQUENCE LISTING (15 sequences)

SEQ ID NO: 1 - DNA, 17 bp, Artificial
  synthesized
atcaagaaggtggtgaa

SEQ ID NO: 2 - DNA, 33 bp, Artificial
  synthesized
tgctcagtgtagcccattcaccaccttcttgat

SEQ ID NO: 3 - DNA, 16 bp, Artificial
  synthesized
tgggctacactgagca

SEQ ID NO: 4 - DNA, 36 bp, Artificial
  synthesized
tgactgcgagtagtagccattcaccaccttcttgat

SEQ ID NO: 5 - DNA, 19 bp, Artificial
  synthesized
tggctactactcgcagtca

SEQ ID NO: 6 - DNA, 29 bp, Artificial
  synthesized
tctcaggtaccattcaccaccttcttgat

SEQ ID NO: 7 - DNA, 12 bp, Artificial
  synthesized
tggtacctgaga

SEQ ID NO: 8 - DNA, 17 bp, Artificial
  synthesized
ccaccgtttttcctcag

SEQ ID NO: 9 - DNA, 33 bp, Artificial
  synthesized
tgctcagtgtagcccactgaggaaaaacggtgg

SEQ ID NO: 10 - DNA, 36 bp, Artificial
  synthesized
tgactgcgagtagtagccactgaggaaaaacggtgg

SEQ ID NO: 11 - DNA, 29 bp, Artificial
  synthesized
tctcaggtaccactgaggaaaaacggtgg

SEQ ID NO: 12 - DNA, 17 bp, Artificial
  synthesized
atcttaaatcgcaaggt

SEQ ID NO: 13 - DNA, 33 bp, Artificial
  synthesized
tgctcagtgtagcccaaccttgcgatttaagat

SEQ ID NO: 14 - DNA, 36 bp, Artificial
  synthesized
tgactgcgagtagtagccaaccttgcgatttaagat

SEQ ID NO: 15 - DNA, 29 bp, Artificial
  synthesized
tctcaggtaccaaccttgcgatttaagat

(19) United States
(12) Patent Application Publication
(10) Pub. No.: US 2021/0285030 A1
(43) Pub. Date: Sep. 16, 2021

(54) KIT AND METHOD FOR DETECTING DROPLET DRIFT OR DEPOSITION CHARACTERISTICS OF SPRAY

(71) Applicant: CHINA AGRICULTURAL UNIVERSITY, Beijing (CN)

(72) Inventors: Zhenhua ZHANG, Beijing (CN); Zongyang LI, Beijing (CN); Xuemin WU, Beijing (CN); Jianli SONG, Beijing (CN); Yang LIU, Beijing (CN); Liu ZHU, Beijing (CN); Sen PANG, Beijing (CN); Xiongkui HE, Beijing (CN); Xuefeng LI, Beijing (CN)

(21) Appl. No.: 17/214,739

(22) Filed: Mar. 26, 2021

Related U.S. Application Data
(63) Continuation-in-part of application No. PCT/CN2019/108038, filed on Sep. 26, 2019.

(30) Foreign Application Priority Data
Sep. 26, 2018 (CN) 201811120814.3

(51) Int. Cl. C12Q 1/6816; C12Q 1/6876; G01N 21/78

(52) U.S. Cl. CPC C12Q 1/6816; G01N 21/78; C12Q 1/6876

(57) ABSTRACT

A kit is employed in the method for detecting droplet drift or deposition characteristics of spray. The detection kit includes detection membranes carrying immobilized probes, transition probes capable of specifically binding to the immobilized probes, and biotinylated chromogenic probes capable of specifically binding to the transition probes. The transition probes are added to the agricultural spray as tracers. After spraying, the sprayed transition probes specifically bind to the immobilized probes on the detection membranes. The biotinylated chromogenic probes are added to bind to the transition probes through hybridization. After the chromogenic treatment, the deposition volume of droplets is determined according to the color depth, and the droplet parameters are determined according to the position and size of chromogenic spots. The method can qualitatively detect the droplet drift or deposition distribution of spray, and to determine the droplet drift or deposition volume and the droplet coverage density and droplet size.

### CROSS-REFERENCE TO RELATED APPLICATIONS

The present application is a continuation in part application of PCT international application no. PCT/CN2019/108038, filed on Sep. 26, 2019, which claims the priority of Chinese Patent Application No. 2018111208143 filed on Sep. 26, 2018 with China National Intellectual Property Administration, and entitled “KIT AND METHOD FOR DETECTING DROPLET DRIFT OR DEPOSITION CHARACTERISTICS OF SPRAY”, which is incorporated herein by reference in its entirety.

### INCORPORATION OF SEQUENCE LISTING

This application contains a sequence listing submitted in Computer Readable Form (CRF). The CFR file containing the sequence listing entitled “PA150-0107_ST25.txt”, which was created on Mar. 26, 2021, and is 2,957 bytes in size. The information in the sequence listing is incorporated herein by reference in its entirety.

### TECHNICAL FIELD

The present invention relates to the technical field of spray droplet detection, in particular to a reverse dot blot kit and a detection method developed for detecting drift and deposition characteristics of spray in agricultural, including the analysis of the size and distribution of the droplets and the quantitative detection of droplet drift and deposition.

### BACKGROUND

In modern agriculture, the key points for ensuring a stable or increased yield of agricultural products lie in the control of crop pests and the spraying of liquid fertilizers. Spraying is one of the most common application methods in the use of pesticides and fertilizers, such as insecticides, fungicides, and liquid fertilizers. Taking pesticide spraying as an example, the application of insufficient amount of pesticide cannot achieve the control effect of killing pests, leading to a reduction in agricultural products or even no harvest, and causing the waste of human resources. However, the excessive amount of pesticide will cause environmental pollution and resource waste, and the excessive pesticide residues in food are then enriched continuously along with the food chain and are harmful to people's health. Therefore, the application of reasonable amount of pesticides becomes one of important factors to ensure a stable or increased yield of agricultural products. The detection of droplet deposition characteristics of pesticide spray provides guidelines for the rational use of pesticides, and the determination of deposition distribution of the pesticide droplets on a target during the spraying process is an important indicator for evaluating the pesticide spraying efficiency. Common methods for determining the drift or deposition distribution of pesticide spray droplets include direct detection method, tracer method and direct droplet observation method. Among them, the direct detection method and the tracer method can only be used for quantitative detection of the deposition volume of the pesticide liquid, but cannot provide information such as the droplet coverage density and the droplet size of the pesticide liquid; in contrast, the direct droplet observation method can only obtain information such as the droplet coverage density and droplet size of the pesticide liquid, but cannot perform quantitative detection.

The direct detection method usually comprises the following steps: taking crops as targets receiving pesticide droplets, directly spraying pesticides on the target crops, collecting crop samples after spraying, directly detecting the amount of pesticide active ingredient on the targets by instruments such as HPLC-MS and GC-MS, and then obtaining the deposition distribution of the pesticide droplets. Although this method has high accuracy, but it required expensive instruments, has slow testing speed, and it is not favorable for long-distance testing due to the low temperature storage condition for the collected samples.

The tracer method usually comprises the following steps: adding tracers into the pesticide liquid, analyzing and determining the amount of tracers on the target by an instrument, and then calculating the pesticide deposition volume on the target. Common tracers include water-soluble dyes such as tartrazine and allura red and fluorescent tracers such as brilliant sulphoflavine (BSF) and pyranin. This method has the advantages of rapid detection, cost-efficiency and low requirement on reagent storage, and is one of common methods for detecting pesticide spray deposition. However, the precision of the method is susceptible to the properties of tracers and thus is not good, and may cause color contamination for the crops and the operators as tracers are usually colored agents.

The direct droplet observation method usually comprises the following steps: taking water sensitive paper, oil sensitive paper, Kromekote® cards and other test cards as targets for receiving droplets, and providing information such as the size and the coverage density of droplets by an image processing method; or directly observing properties of droplets using instruments such as a laser particle size analyzer. Water sensitive paper method is a commonly used method in the industry. This method can observe the deposition and distribution of droplets on site, but the water sensitive paper changes color when contacting with moisture and is thus susceptible to the environment. The method is inapplicable in rainy days or in a condition of high humidity, and is incapable of quantitative analysis of droplets.

All the three methods can only detect the deposition distribution of pesticide droplets qualitatively or quantitatively, but cannot simultaneously determine information such as the droplet deposition on the target, the coverage density and size of the droplets. Accordingly, a method combining the direct droplet observation with the tracer method is generally adopted to comprehensively determine and evaluate pesticide spray efficiency. As the water sensitive paper method is greatly influenced by the environmental humidity, the spray droplet deposition volume cannot be accurately detected. The detection methods based on chromatography and luminosity require expensive instruments, sample pretreatment and professional operation technicians. However, in actual spraying operations, timely and quick determination of the deposition distribution of pesticide on the plant leaf surfaces are required. Therefore, it is urgent to develop a rapid, low-cost, high-accuracy detection method to qualitatively and quantitatively evaluating droplet deposition of spray.

Reverse dot blot (RDB) is a common DNA detection technique, and utilizes the specific binding of DNA sequence, immobilizes the complementary strand of a target DNA on a substrate; the complementary strand binds to a DNA sample to be detected after extraction and amplification, and captures a biotinylated DNA to be detected after amplification, thus achieving the detection of the sample. This technique has been used in the fields of pathogens, tumor gene detection, virus typing, gene mutation and the like owning to its rapidness and simplicity, high sensitivity, high specificity and good accuracy. However, the current application of this technique is mainly focused on the detection of target DNA sequences after amplification, but the application of an artificially synthesized single-stranded deoxyribonucleic acid with a characteristic sequence as a tracer, especially as a tracer in the agricultural spraying process, has not been reported. The existing RDB technique usually adopts a two-stage mode, namely, the DNA to be detected is amplified and labeled with biotin or enzyme on a short nucleic acid chain side, and when the amplified DNA to be detected is combined with a complementary strand on a substrate, direct color development can be realized. However, the application of short DNA strand labeled with biotin or enzyme as a tracer in spraying detection costs a lot, which undoubtedly limits the popularization and application of RDB techniques. It is urgent to develop a low-cost and easy-to-operate method that is capable of simultaneously determining information such as the pesticide deposition on the target, and the coverage density and size of the droplets accurately and quickly.

### SUMMARY

The objective of the present invention is to develop a kit for qualitatively and quantitatively detecting droplet drift or deposition distribution of sprays of pesticides, liquid fertilizers or other liquid formulations by reverse dot blot, thereby realizing rapid, accurate, easy-to-operate and low-cost detection of distribution characteristics of agricultural spray droplets.

The present invention develops a method for detecting droplet drift or deposition distribution characteristics of spray, and the specific process is shown in FIG. 1. In the present invention, “three-stage” reverse dot blot is adopted: firstly, immobilizing “immobilized probes” on membrane substrates by chemical bonding, then adding “transition probes” as tracers to pesticide, liquid fertilizer or other liquid formulations to be sprayed, and after spraying, performing complementary pairing of part of bases of the “immobilized probes” and the “transition probes” to capture the “transition probes”; after recovering the membrane, adding “chromogenic probes” labeled with biotin or enzyme to interact with the other part of bases of the “transition probes”; after catalytic chromogenic reaction through the signal amplification of the enzyme, observing information such as the size, distribution, and coverage rate of droplets; finally, obtaining a digital image file by means such as photographing or scanning, reading gray difference by an image processing software, and calculating the deposition volume of droplets according to a standard curve. Specifically, the method for detecting droplet drift or deposition volume of spray provided herein comprises the following process: preparation of detection membranes, preparation and spraying of the spray liquid, chromogenic treatment and detection.

The present invention firstly provides a method for detecting droplet drift or deposition characteristics of spray, comprising the following steps: adding transition probes as tracers to pesticide liquid; after spraying the pesticide or liquid fertilizer or other liquid formulations, specifically binding the transition probes to the immobilized probes on detection membranes, wherein the detection membranes are substrates carrying the immobilized probes; and detecting the transition probes on the detection membranes to determine the droplet drift or deposition of the spray.

The transition probes and the immobilized probes are single-stranded deoxyribonucleic acids with characteristic sequences, wherein the length of the immobilized probe may be 12-25 nt, preferably 18-20 nt; one end of the immobilized probe is amino-modified and covalently bind to an exposed carboxyl of the substrate.

In order to reduce costs, in the present invention, “three-stage” reverse dot blot is adopted. In the “three-stage” reverse dot blot, the transition probes are not biotinylated; the transition probes specifically bind to the immobilized probes on detection membranes, and then chromogenic probes labeled with biotin bind to the transition probes through hybridization; after chromogenic treatment, the spray deposition are determined according to the color depth, and the coverage rate and the volume of droplets are determined according to the position and the size of chromogenic spots.

In one embodiment of the present invention, the complementary pairing region of the chromogenic probe and the transition probe is of 15-40 nt. If the immobilized probe is 5′-labeled, the chromogenic probe is 3′-biotinylated; and if the immobilized probe is 3′-labeled, the chromogenic probe is 5′-biotinylated. The complementary pairing region of the transition probe and the immobilized probe is of 15-25 nt.

In the preparation of detection membranes of the present invention, the major reagents used include: 0.1-0.3 M (preferably 0.1 M) HCl solution, 10-20% (preferably 15%) EDC (1-(3-dimethylaminopropyl)-3-ethylcarbodiimide) solution, 0.025-0.2 μM (preferably 0.03 μM) immobilized probe solution, 0.3-1.0 M (preferably 0.5 M) NaHCO₃ solution, and 0.05-0.5 M (preferably 0.2 M) NaOH solution.

The substrate includes but is not limited to a nitrocellulose membrane, a nylon membrane, a carboxylated organic glass film and a carboxylated polypropylene plastic film.

The detection membrane is prepared according to the following method: acquiring a substrate of a required area, treating the substrate with 0.1-0.3 M HCl, and washing; incubating the substrate in 10-20% EDC solution and washing; incubating the substrate in 0.3-1.0 M NaHCO₃ solution containing 0.025-0.2 μM immobilized probe; and incubating the substrate in 0.05-0.5 M NaOH solution, washing and drying; wherein the amino or carboxyl of the substrate is exposed.

Preferably, the detection membrane is prepared according to the following method: acquiring a substrate of a required area, treating the substrate with 0.1 M HCl, and washing; incubating the substrate in 15% EDC solution for 0.5-1 h and washing; incubating the substrate in 0.5 M NaHCO₃ solution containing 0.03 μM immobilized probe for 10-20 min; and incubating the substrate in 0.2 M NaOH solution for 5-15 min, washing and drying.

In the preparation of spray liquid, the spray liquid mainly comprises: 0-60% of pesticide formulation or liquid fertilizer (or water), 0.025-0.1 μM (preferably 0.06 μM) transition probe, 0-0.045 mol/L ion buffer, and 0-0.15% of surfactant (if pesticide or fertilizer dilution is used, the transition probe can be directly added because the pesticide or fertilizer itself may contain surfactant and ion buffer; if water is used as the spray liquid, a certain amount of ion buffer and surfactant should be added). Major formulations include water-based formulation and oil-based formulation.

The pesticide formulations includes water-based formulation, oil-based formulation, wettable powder, microcapsule, water-based suspension, oil-based suspension and the like, wherein the pesticide includes insecticides, fungicides, herbicides, acaricides, nematicides and the like.

The liquid fertilizer include solution, suspension, foliar fertilizer and the like, wherein the fertilizer includes any one of a nitrogenous fertilizer, a phosphate fertilizer or a potash fertilizer, or a compound fertilizer comprising two or more thereof.

The transition probe may be a single-stranded deoxyribonucleic acid with a characteristic sequence of 24-50 nt (preferably 36-40 nt).

The ion buffer is a buffer prepared from one or more inorganic salts and/or organic salts, wherein the anion of the solution is one or more of carbonate, bicarbonate, phosphate, hydrogen phosphate, dihydrogen phosphate, citrate, dihydrogen citrate and the like, and the cation is one or more of potassium ion, sodium ion, lithium ion, calcium ion and the like.

The surfactant is one or more of sodium alkyl sulfonate, nekal, tea seed powder, Gleditsia sinensis extract powder, SDS (sodium dodecyl sulfate), Morwet EFW (sodium butylnaphthalene sulfonate), TERWET 1004 and the like.

In the chromogenic treatment, the reagents used include: hybridization buffer, washing buffer, 0.05-0.20 μM chromogenic probe solution, catalase solution, and TMB (3,3′,5,5′-tetramethylbenzidine) single-component solution.

The hybridization buffer mainly comprises 0.02-0.045 mol/L ion buffer and 0.06-0.15% surfactant.

The washing buffer mainly comprises 5.0-10.0 mol/L ion buffer and 0.02-0.20% of surfactant.

The ion buffer is a buffer prepared from one or more inorganic salts and/or organic salts, wherein the anion of the solution is one or more of carbonate, bicarbonate, phosphate, hydrogen phosphate, dihydrogen phosphate, citrate, dihydrogen citrate and the like, and the cation is one or more of potassium ion, sodium ion, lithium ion, and calcium ion.

The surfactant is one or more of sodium alkyl sulfonate, nekal, tea seed powder, Gleditsia sinensis extract powder, SDS (sodium dodecyl sulfate), Morwet EFW (sodium butylnaphthalene sulfonate), and TERWET 1004.

The chromogenic probe of the present invention is not particularly limited, and may be a single-stranded deoxyribonucleic acid with a characteristic sequence of 12-25 nt, for example, a single-stranded deoxyribonucleic acid with a characteristic sequence of 18-20 nt.

The TMB single-component solution mainly comprises: 0.5-2.0 mM (preferably 1.0 mM) TMB, 0.5-2.0 mM (preferably 1.0 mM) oxidant, 150-300 mM (preferably 200 mM) ion buffer, and 0.1-0.5 mM stabilizer. The specific preparation process is as follows: solution a: dissolving certain amounts of TMB and stabilizer in DMSO; solution b: preparing an ion buffer with deionized water, adding oxidant, and adjusting the pH to 4.0-6.0 with hydrochloric acid; and mixing solution a and solution b in a certain ratio to give the TMB single-component solution before use. The ratio of solution a to solution b is not particularly limited, and may be adjusted by those skilled in the art according to the conditions known in the art.

The oxidant is one or more of hydrogen peroxide, urea-hydrogen peroxide, peroxyacetic acid, tert-butyl hydroperoxide, and dimethyl dioxirane.

The ion buffer is a buffer prepared from one or more inorganic salts and/or organic salts, wherein the anion of the solution is one or more of carbonate, bicarbonate, phosphate, hydrogen phosphate, dihydrogen phosphate, citrate, dihydrogen citrate and the like, and the cation is one or more of potassium ion, sodium ion, lithium ion, calcium ion and the like.

The stabilizer is one or more of sodium borohydride, sodium cyanoborohydride, tetrabutylammonium borohydride (TBABH), lithium tri-sec-butylborohydride, lithium borohydride and the like.

The specific processes are as follows:

1) Preparation of detection membranes: treating the substrate with HCl for 1-5 min and washing; incubating the substrate in EDC for 0.5-1 h and washing; incubating the substrate in NaHCO₃ solution containing an immobilized probe for 10-20 min; and incubating the substrate in NaOH solution for 5-15 min, washing, drying, and cutting into a required area to obtain the detection membrane carrying immobilized probe.

2) Preparation and spraying of spray liquid: adding a pesticide dilution, a liquid fertilizer or water into a container, then adding transition probe and water, and mixing well, and finally adding a surfactant and an ion buffer based on the requirements of the pesticide formulation or spraying equipment to prepare the transition probe spray liquid; after spraying, recovering the detection membranes for chromogenic treatment.

3) Chromogenic treatment: incubating the detection membranes sprayed with the transition probe spray liquid in a hybridization buffer at 30-40° C. for 25-40 min, then transferring the detection membranes into 50 mL of hybridization buffer for 2 min for pretreatment; transferring the detection membranes into a hybridization buffer containing chromogenic probes for chromogenic reaction at 30-40° C. for 5-15 min; washing the detection membranes with 50 mL of washing buffer for 3 times; washing the detection membranes with a hybridization buffer for 2 min; adding 15 μL of catalase solution to a hybridization buffer to prepare an enzyme solution, then incubating the detection membranes in the enzyme solution for enzyme-linked reaction at 37° C. for 15-20 min; after the reaction is completed, washing the detection membranes with a hybridization buffer, and adding a TMB single-component solution for chromogenic reaction, wherein the TMB single-component solution is catalyzed by catalase binding to the detection membranes, resulting in chromogenic spots on the detection membranes; after 3 min, washing the membranes with water to terminate the reaction and drying, directly observing information such as the distribution and the size of droplets through the chromogenic reaction; obtaining an image file by photographing or scanning, reading the gray values of unit areas by an image processing software (e.g., Photoshop, Image J, and the like), and calculating a total gray value of a selected area; finally calculating the deposition volume according to a standard curve.

4) Detection: firstly, establishing a standard curve: applying 0.5 μL of the transition probe spray liquid (namely the spray liquid prepared in step 2) on 5 detection membranes carrying immobilized probes using a pipette to form 1, 2, 3, 4 or 5 spots, wherein the volumes of the probe solutions on the 5 detection membranes are 0.5 μL, 1.0 μL, 1.5 μL, 2.0 μL and 2.5 μL, respectively; taking another detection membrane as background; performing chromogenic treatment according to step 3); obtaining an image file by photographing or scanning, reading the gray values of unit areas by an image processing software (e.g., Photoshop, Image J, and the like), and calculating a total gray value of a selected area; finally, plotting a standard curve with total gray value as the ordinate against the volume of the spray liquid as the abscissa, and calculating a corresponding linear equation.

According to the present invention, the catalase is streptavidin-labeled horseradish catalase.

Based on the detection method disclosed herein, the present invention also provides a kit for detecting droplet drift or deposition volume of spray, comprising detection membranes, transition probes and chromogenic probes; wherein, the detection membrane is a substrate carrying the immobilized probes, the length of the immobilized probe is 12-25 nt, one end of the immobilized probe is amino-modified and covalently binds to an exposed carboxyl of the substrate; and the substrate is amino-exposed or carboxyl-exposed; the length of the transition probe is 24-50 nt; the 3′ or 5′ end of the chromogenic probe is labeled with biotin, and the chromogenic probe can specifically bind to the transition probe but cannot specifically bind to the immobilized probe.

The kit also comprises streptavidin-labeled horseradish catalase and TMB single-component solution.

In the present invention, “three-stage” reverse dot blot hybridization is adopted, which comprises the following steps: adding transition probes not labeled with biotin or enzyme as a tracer to the spray liquid, and after spraying, specifically binding the transition probes to the immobilized probes on detection membranes; binding the chromogenic probes labeled with biotin to the transition probes through hybridization, performing chromogenic treatment according to the streptavidin-catalase-TMB chromogenic reaction principle, determining the droplet deposition volume according to the color depth, and determining droplet deposition parameters according to the position and the size of chromogenic spots. The method can qualitatively and quantitatively detect the droplet deposition volume of pesticide spray, and is accurate and efficient, fast and convenient, easy-to-operate, and low-cost. The detection method disclosed herein has the following beneficial effects: (1) it solves the problem that the water sensitive paper cannot work in a humid environment in a field test; (2) it avoids the influence of droplet superposition and rolling behavior on the determination precision, and thus can quantitatively determine the results; (3) it solves the problem of color contamination of water-soluble dyes and fluorescent tracers to environment by introducing transition probes as tracers, which are colorless and tasteless and pose no pollution to the environment; (4) it is based on the molecular hybridization, and thus can improve the sensitivity greatly, detect probes as low as 5 pg owning to the dot blot hybridization, is capable of replacing the fluorescent tracer for deposition determination, and solve the problem that the fluorescent tracer is easy to be photolyzed, as compared with the conventional method for evaluating pesticide spraying efficacy; (5) it is capable of simultaneously performing qualitative and quantitative detections of the droplet deposition, and solves the problems that the water sensitive paper can only be used for determining the amount and coverage rate of the droplets in the chromogenic treatment, and that tracer method or direct detection method can only be used for measuring the deposition volume of the droplets.

In a word, the detection method disclosed herein can be used for qualitatively detecting the deposition distribution of the pesticide droplets, and can also be used for quantitatively determining the spray droplet deposition, as well as the coverage density and size of the droplets; it can accurately guide the pesticide application, improve the pesticide utilization rate, reduce environmental pollution, and improve pesticide application technical system; and thus will have a good application prospect in the market.

### BRIEF DESCRIPTION OF THE DRAWINGS

FIG. 1 is a schematic flow chart of the three-stage reverse dot blot for detecting the droplet deposition of spray.

FIG. 2 shows the result of the establishment of the standard curve according to the detection method of the present invention.

FIG. 3 shows the detection result of the sensitivity of the droplet drift of pesticide spray according to the detection method of the present invention.

FIG. 4A shows the result of the droplet amount of spray boom track system-simulated field spraying according to detection method of the present invention, and FIG. 4B shows the result of the coverage rate of droplets of spray boom track system-simulated field spraying according to detection method of the present invention.

FIG. 5 shows the result of the droplet deposition of spray boom track system-simulated field spraying according to detection method of the present invention.

FIG. 6 shows the detection results of the coverage rate, amount and deposition volume of the droplets per unit area after unmanned aerial vehicle (UAV) field spraying according to detection of the present invention.

FIG. 7 is a photograph of the arrangement of the detection membranes in the deposition distribution test on rice canopy in Example 6.

FIG. 8 shows the detection result of the spray droplet deposition in Example 6.

FIG. 9 is a photograph of the arrangement of the detection membranes in the deposit distribution test of cotton defoliant in Example 7.

FIG. 10 shows the detection result of the spray droplet deposition in Example 7.

FIG. 11 shows the detection result of spray droplet drift in an integrated rice-crayfish farming system in Example 8.

### DETAILED DESCRIPTION

The present invention will be further illustrated below with reference to examples, which should not be construed as limiting the present invention. Modifications or substitutions to the methods, procedures or conditions of the present invention may be implemented without departing from the spirit and scope of the present invention.

Unless otherwise specified, the techniques used in the examples are conventional techniques well known to those skilled in the art.

### Example 1: Establishment of Method for Detecting Droplet Drift or Deposition of Spray

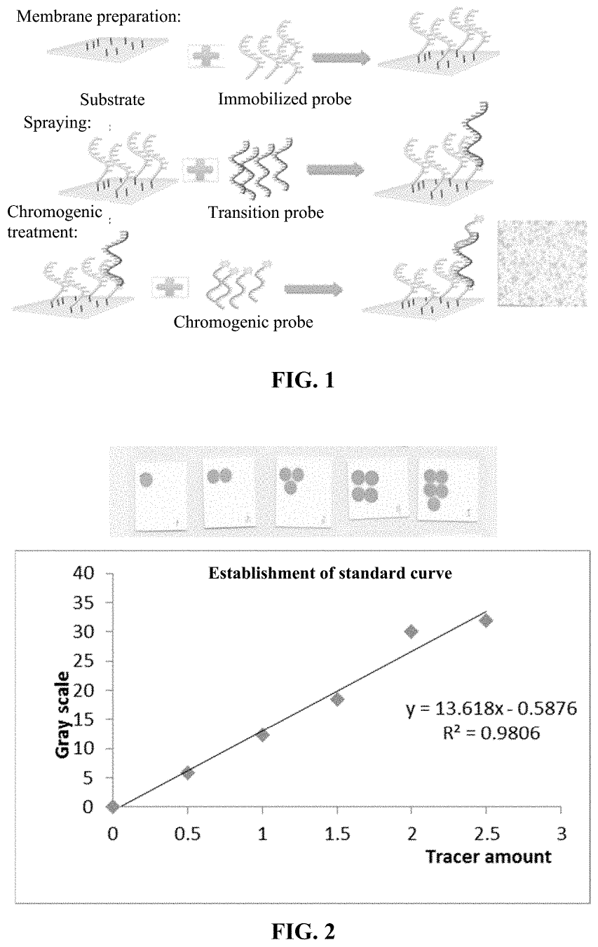

In the present invention, “three-stage” reverse dot blot hybridization is adopted, and the process is shown in FIG. 1. Firstly, “immobilized probes” were immobilized on substrates by chemical bonding, then “transition probes” as tracers were added to pesticide, liquid fertilizer or other liquid formulations to be sprayed; and after spraying, complementary pairing of part of bases of the “immobilized probes” and the “transition probes” was performed to capture the “transition probes”. After the membrane was recovered, “chromogenic probes” labeled with biotin or enzyme were added to interact with the other part of bases of the “transition probes”. After the chromogenic reaction was catalyzed through the signal amplification of the enzyme, information such as the size, distribution, and coverage rate of droplets was observed. Finally, a digital image file was obtained by means such as photographing or scanning, the gray difference was read by an image processing software, and the spray droplet deposition was calculated according to the standard curve.

1. Determination of Probes

The length of the immobilized probe was 12-25 nt, preferably 18-20 nt. One end of the immobilized probe was amino-modified, and the other end was covalently bound to an exposed carboxyl of the substrate.

The transition probe was a single-stranded deoxyribonucleic acid with a characteristic sequence of 24-50 nt (preferably 36-40 nt) without biotinylation. The complementary pairing region of the transition probe and the immobilized probe was of 15-25 nt.

The chromogenic probe was a single-stranded deoxyribonucleic acid with a characteristic sequence of 12-25 nt (preferably 18-20 nt). The complementary pairing region of the chromogenic probe and the transition probe was of 15-40 nt. If the immobilized probe was 5′-labeled, the chromogenic probe was 3′-biotinylated; and if the immobilized probe was 3′-labeled, the chromogenic probe was 5′-biotinylated.

The chromogenic probe could specifically bind to the transition probe but could not specifically bind to the immobilized probe. The sequences of three probes in Table 1 are exemplary probe sequences. In addition to the nucleotide sequences of the probes in Table 1, any single-stranded deoxyribonucleic acid sequence that satisfies the above requirements can be used for the techniques in the present invention.

TABLE 1

| | | |
|---|---|---|
| 1 | Immobilized probe 1 | 5′-NH₂- ATCAAGAAGGTGGTGAA -3′ |
| | Transition probe 1 | 5′- TGCTCAGTGTAGCCCATTCACCACCTTCTTGAT -3′ |
| | Chromogenic probe 1 | 5′ TGGGCTACACTGAGCA -Biotin-3′ |
| 2 | Immobilized probe 1 | 5′-NH₂- ATCAAGAAGGTGGTGAA -3′ |
| | Transition probe 1-2 | 5′- TGACTGCGAGTAGTAGCCATTCACCACCTTCTTGAT -3′ |
| | Chromogenic probe 2 | 5′ TGGCTACTACTCGCAGTCA -Biotin-3′ |
| 3 | Immobilized probe 1 | 5′-NH₂- ATCAAGAAGGTGGTGAA -3′ |
| | Transition probe 1-3 | 5′- TCTCAGGTACCA TTCACCACCTTCTTGAT -3′ |
| | Chromogenic probe 3 | 5′ TGGTACCTGAGA -Biotin-3′ |
| 4 | Immobilized probe 2 | 5′-NH₂-CCACCGTTTTTCCTCAG-3′ |
| | Transition probe 2 | 5′-TGCTCAGTGTAGCCCACTGAGGAAAAACGGTGG -3′ |
| | Chromogenic probe 1 | 5′ TGGGCTACACTGAGCA -Biotin-3′ |
| 5 | Immobilized probe 2 | 5′-NH₂-CCACCGTTTTTCCTCAG-3′ |
| | Transition probe 2-2 | 5′- TGACTGCGAGTAGTAGCCACTGAGGAAAAACGGTGG -3′ |
| | Chromogenic probe 2 | 5′ TGGCTACTACTCGCAGTCA -Biotin-3′ |
| 6 | Immobilized probe 2 | 5′-NH₂-CCACCGTTTTTCCTCAG-3′ |
| | Transition probe 2-3 | 5′- TCTCAGGTACCA CTGAGGAAAAACGGTGG -3′ |
| | Chromogenic probe 3 | 5′ TGGTACCTGAGA -Biotin-3′ |
| 7 | Immobilized probe 3 | 5′-NH₂-ATCTTAAATCGCAAGGT-3′ |
| | Transition probe 3 | 5′- TGCTCAGTGTAGCCCAACCTTGCGATTTAAGAT -3′ |
| | Chromogenic probe 1 | 5′ TGGGCTACACTGAGCA -Biotin-3′ |
| 8 | Immobilized probe 3 | 5′-NH₂-ATCTTAAATCGCAAGGT-3′ |
| | Transition probe 3-2 | 5′- TGACTGCGAGTAGTAGCCAACCTTGCGATTTAAGAT -3′ |
| | Chromogenic probe 2 | 5′ TGGCTACTACTCGCAGTCA -Biotin-3′ |
| 9 | Immobilized probe 3 | 5′-NH₂-ATCTTAAATCGCAAGGT-3′ |
| | Transition probe 3-3 | 5′- TCTCAGGTACCA ACCTTGCGATTTAAGAT -3′ |
| | Chromogenic probe 3 | 5′ TGGTACCTGAGA -Biotin-3′ |
| 10 | Immobilized probe 1 | 5′-NH₂- ATCAAGAAGGTGGTGAA -3′ |
| | Transition probe 2 | 5′- TGCTCAGTGTAGCCCACTGAGGAAAAACGGTGG -3′ |
| | Chromogenic probe 3 | 5′ TGGTACCTGAGA -Biotin-3′ |

2. Preparation of Detection Membranes

A required area of a nylon membrane enriched with carboxyl on the surface was prepared, treated with 0.1 M HCl, and washed; the membrane was incubated in 15% EDC solution for 1 h and washed; then the membrane was incubated in 0.5 M NaHCO₃ solution containing 0.03 μM immobilized probe (e.g., immobilized probe 1 in Table 1) for 20 min; the treated membrane was incubated in 0.2 M NaOH solution for 15 min, washed and dried. The prepared detection membranes were arranged on spraying targets for collecting spray droplets and the subsequent detection.

3. Preparation and Spraying of Spray Liquid

A spray liquid containing 30 mM trisodium citrate, 0.9% SDS and 0.06 μM transition probe (e.g., transition probe 1 in Table 1) was added to a dosing tank, and after spraying, the detection membranes were recovered for chromogenic treatment.

4. Establishment of Standard Curve

0.5 μL of the transition probe spray liquid was applied on 5 detection membranes carrying immobilized probes using a pipette to form 1, 2, 3, 4 or 5 spots, wherein the volumes of the transition probe solutions on the 5 detection membranes were 0.5 μL, 1.0 μL, 1.5 μL, 2.0 μL and 2.5 μL, respectively. Another detection membrane was taken as background. An image file was obtained by photographing or scanning, the gray values of unit areas were read by an image processing software (e.g., Photoshop, Image J, and the like), and a total gray value of a selected area was calculated. Finally, a standard curve with total gray value as the ordinate against the volume of the spray liquid as the abscissa was plotted, and a corresponding linear equation was calculated. The results are shown in FIG. 2. The linear equation is y=13.618x−0.5876, with a linear correlation coefficient of 0.98, which meets the standard curve requirements for quantitative detection.

5. Chromogenic Treatment

The detection membranes sprayed with the transition probe spray liquid were collected and incubated in 50 mL of hybridization buffer (an aqueous solution containing 30 mmol/L trisodium citrate and 26 mmol/L SDS) at 34° C. for 40 min; the detection membranes were transferred into 50 mL of hybridization buffer for washing for 2 min; the detection membranes were transferred into a hybridization buffer containing chromogenic probes (e.g., chromogenic probe 1 in Table 1) for reaction at 37° C. for 15 min, then were washed 3 times with 50 mL of washing buffer (an aqueous solution containing 7.5 mmol/L trisodium citrate and 6 mmol/L SDS) and washed once with 50 mL of hybridization buffer. 15 μL of catalase solution was added to a hybridization buffer to prepare an enzyme solution, then the detection membranes were incubated in the enzyme solution for enzyme-linked reaction at 37° C. for 20 min; the detection membranes were washed with 50 mL of hybridization buffer, and transferred into a TMB single-component solution for chromogenic reaction, wherein the TMB single-component solution was catalyzed by horseradish peroxidase binding to the detection membranes, resulting in chromogenic spots on the detection membranes. After 3 min, the membranes were washed with water to terminate the reaction, and dried. Information such as the distribution and size of the droplets could be directly observed through the chromogenic reaction. Finally, an image file was obtained by photographing or scanning, the gray values of unit areas were read by an image processing software (e.g., Photoshop, Image J, and the like), a total gray value of a selected area was calculated, and the deposition was calculated according to the standard curve.

### Example 2: Experiment of Droplet Drift of Pesticide Spray

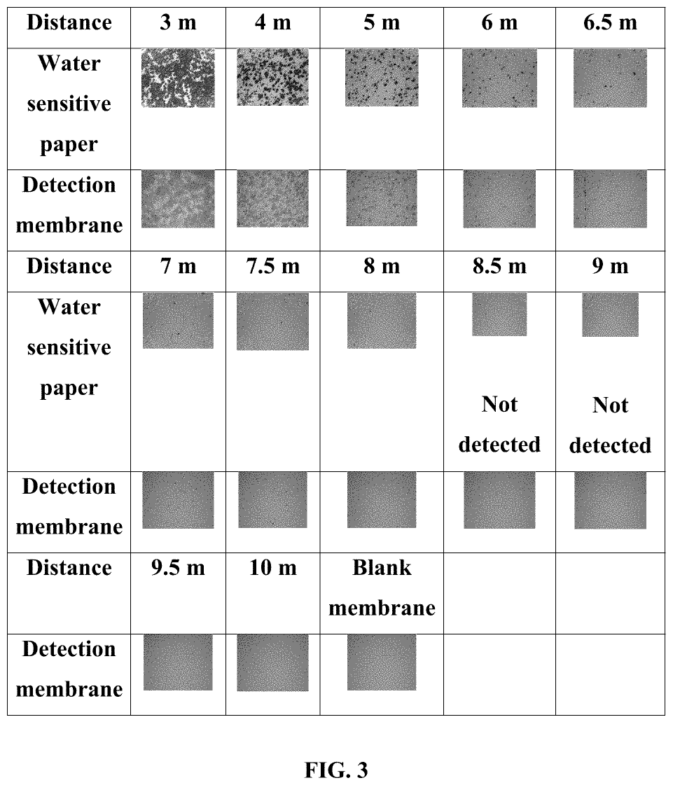

The projection of the nozzle of air-assisted sprayer on the ground was taken as the original point, and sites were taken at 3.0 m, 4.0 m, 5.0 m, 6.0 m, 6.5 m, 7.0 m, 7.5 m, 8.0 m, 8.5 m, 9.0 m, 9.5 m and 10.0 m away from the original point in the Y axis direction. Then one pre-prepared detection membrane (prepared by referring to the preparation of detection membranes in Example 1) and one sheet of water sensitive paper (purchased from Syngenta) were placed at each site, with a cork block serving as a support. At the beginning of the experiment, a baffle was used to block the jet (where the jet liquid was the transition probe spray liquid as described in Example 1, and group 1 in Table 1 was selected as the probes). The baffle was removed when the jet liquid was stable, a stopwatch was used to time the spraying, and the spraying was 30 s in total. The detection membranes and the water sensitive paper were collected. The water sensitive paper directly developed color after spraying; the detection membranes developed color according to the chromogenic treatment as described in Example 1. The results are shown in FIG. 3.

After comparison, it is found that the detection membrane and the water sensitive paper can better reflect information such as the size and the coverage density of droplets at sites of 3-6 m. On this basis, the detection membrane can develop colors of different depth, from which the distribution of droplets can be preliminarily determined. In contrast, the water sensitive paper cannot develop colors of different depth; At sites of 6.5-8 m, the sensitivity of the water sensitive paper is greatly reduced, while the detection membrane can well reflect the condition of droplets. At sites more than 8 m, the water sensitive paper is substantially unable to detect the drop of droplets, while the detection membrane is still able to receive droplets and develop color at sites up to 10 m. This suggests that the detection membrane has lower detection limit and higher sensitivity for qualitative determination of information such as the coverage density and size of the droplets, as compared with the water sensitive paper.

### Example 3: Experiment of Spray Boom Track System-Simulated Field Spraying—Droplet Volume and Coverage Rate

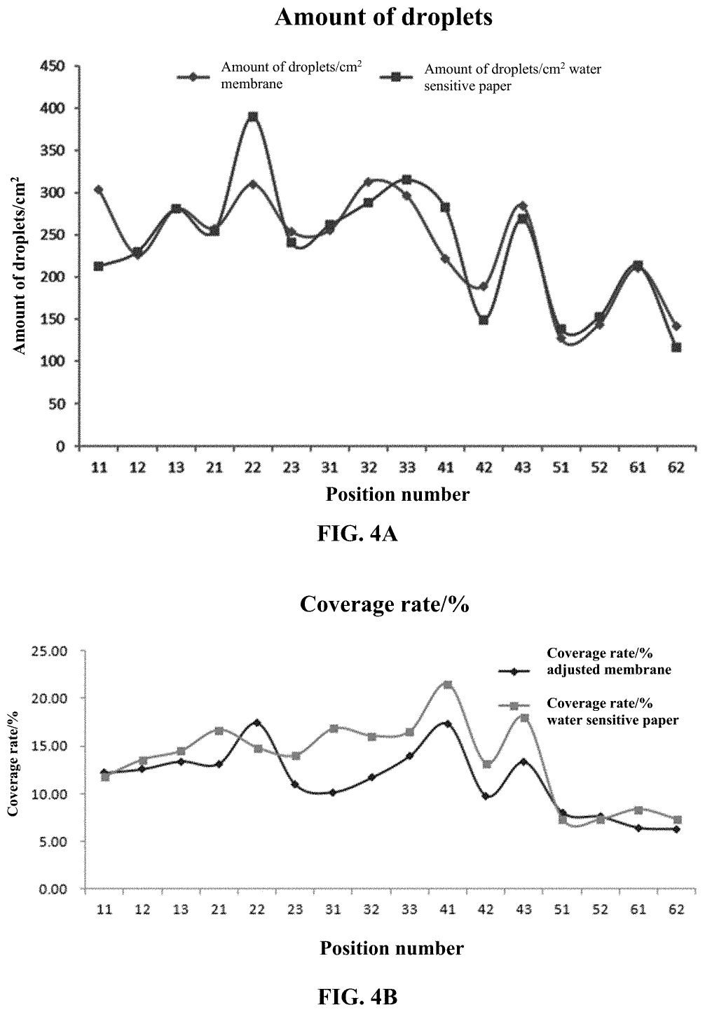

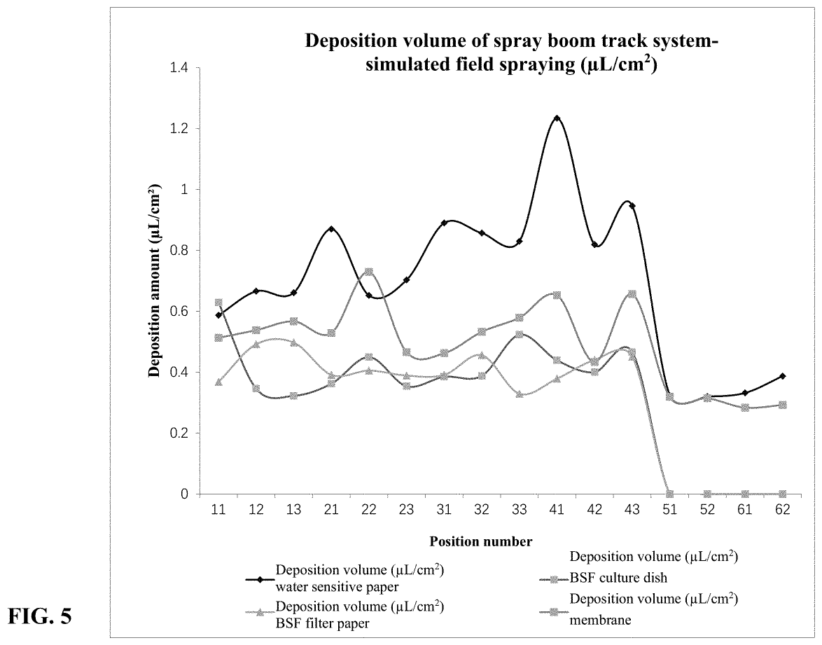

Culture dishes were placed on the iron support below the pathway of the spray boom track system. The culture dishes each contained 2 detection membranes (prepared by referring to Example 1, the results of the 2 detection membranes being averaged as the result of the culture dish) and 1 sheet of water sensitive paper, and 3 culture dishes were placed in total. The culture dishes were placed just in the middle of the pathway of the spray boom track system. Spray (the spray liquid was the transition probe spray liquid as described in Example 1, and group 1 in Table 1 was selected for probes) was applied to the detection membranes under a pressure of 3 bar by the spray boom track system (speed: 5 km/h, height: 0.5 m) equipped with Lechler ST110-03 standard flat spray nozzle. After spraying, the experimental materials were retrieved respectively, and the detection membranes were subjected to chromogenic reactions according to the chromogenic treatment as described in Example 1. The droplet coverage area on the detection membranes and the water sensitive paper was read by an instrument, and the droplet volume and the coverage rate were calculated. The results show that the droplet coverage rate of the detection membrane is consistent with that of the water sensitive paper (see FIG. 4A and FIG. 4B for specific results).

### Example 4: Experiment of Spray Boom Track System-Simulated Field Spraying—Spray Droplet Deposition

Culture dishes were placed on iron support below the pathway of the spray boom track system. The culture dishes each contained 3 detection membranes (prepared by referring to Example 1, the results of the 3 detection membranes being averaged as the result of the culture dish) and 1 sheet of water sensitive paper, and 6 culture dishes were placed in total. The culture dishes were just placed in the middle of the pathway of the crane. Spray (the spray liquid was the transition probe spray liquid as described in Example 1, and group 1 in Table 1 was selected for probes) was applied to the detection membranes under a pressure of 3 bar by the spray boom track system (speed: 5 km/h, height: 0.5 m) equipped with Lechler ST110-03 standard fan-shaped nozzle. After spraying, the experimental materials were retrieved respectively, and the detection membranes were subjected to chromogenic reactions and drying according to the chromogenic treatment as described in Example 1. A digital image was obtained by means such as photographing or scanning, the gray values of unit areas were read by an image processing software (e.g., Photoshop and Image J), and a total gray value of a selected area was calculated. Finally, the spray droplet deposition was calculated according to the standard curve.

Culture dishes were placed on iron support below the pathway of the spray boom track system. One culture dish containing a sheet of filter paper with a diameter of 9 cm in diameter was placed, followed by one empty culture dish at an interval, and a total of 8 culture dishes were placed in this order. The culture dishes were placed just in the middle of the pathway of the spray crane. Spray (the spray liquid was the transition probe spray liquid containing 1 g/L BSF as described in Example 1) was applied to sample membranes under a pressure of 3 bar by the spray boom track system (speed: 5 km/h, height: 0.5 m) equipped with Lechler ST110-03 standard flat spray nozzle. After spraying, the experimental materials were retrieved, respectively. The empty culture dishes were washed with deionized water (10 mL), the resulting solutions were poured into valve bags, and the fluorescence value of the solution in each valve bag was determined by a fluorescence spectrometer after 10 min. A filter paper section was added into each valve bag, the latter was then added with deionized water (10 mL) and shaken for 10 min, and the fluorescence value of the solution in each valve bag was determined by a fluorescence spectrometer. Finally, the deposition volume was calculated according to the standard curve. The results are shown in FIG. 5. The results show that the detection membranes prepared by the reverse dot blot can obtain consistent results with the conventional method in terms of the determination of the amount, coverage area and deposition volume of the droplets. By using single-stranded deoxyribonucleic acids with characteristic sequences as tracers, as compared with traditional tracers, the dosing concentration of the detection membranes used are greatly reduced, and the color contamination risk is avoided.

### Example 5: Experiment of Unmanned Aerial Vehicle-Simulated Field Spraying

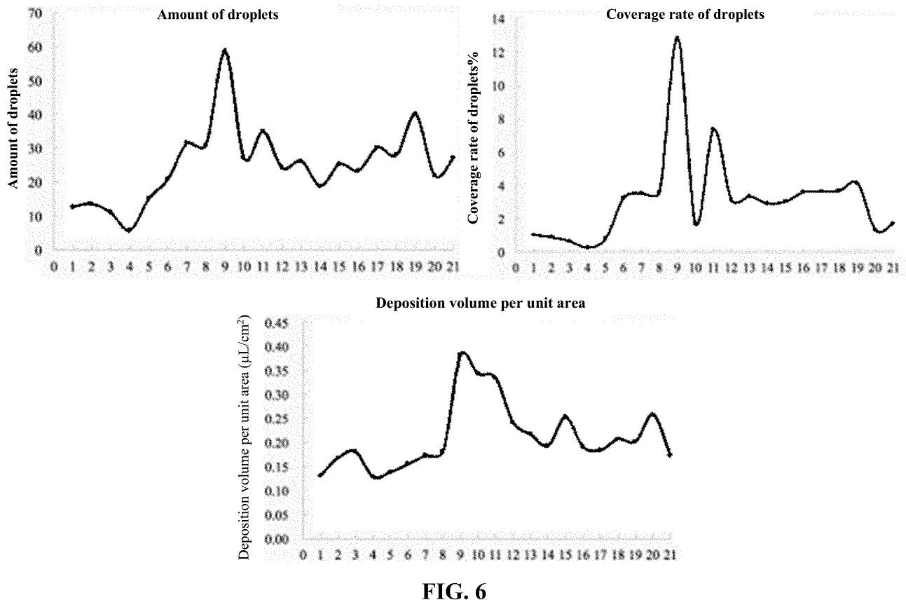

A culture dish was placed every 0.5 m in the direction perpendicular to the pathway of the multi-rotor unmanned aerial vehicle (UAV) sprayer. The culture dishes each contained 3 detection membranes (prepared by referring to Example 1, the results of the 3 detection membranes being averaged as the result of the culture dish), 10 culture dishes were placed in total. Spray was applied to sample membranes under a pressure of 3 bar by the UAV equipped with Lechler LU 120-015 universal flat fan-shaped spray nozzle (height: 3 m, speed: 5 m/s, and spray rod length: 2.0 m). After spraying, the experimental materials were retrieved respectively, and the detection membranes were subjected to chromogenic reactions and drying according to the chromogenic treatment as described in Example 1 to calculate the coverage area and coverage rate of droplets collected by different detection membranes. A digital image was obtained by means such as photographing or scanning, the gray values of unit areas were read by an image processing software (e.g., Photoshop and Image J), and a total gray value of a selected area was calculated. Finally, the deposition volume was calculated according to the standard curve. The results are shown in FIG. 6. The results show that this technique can be used for detecting the droplets of UAV spraying. In addition, the drift and deposition during the spraying process can be well reflected on the amount, coverage rate and deposition volume of the droplets.

### Example 6: Deposition Distribution Test on Rice Canopy

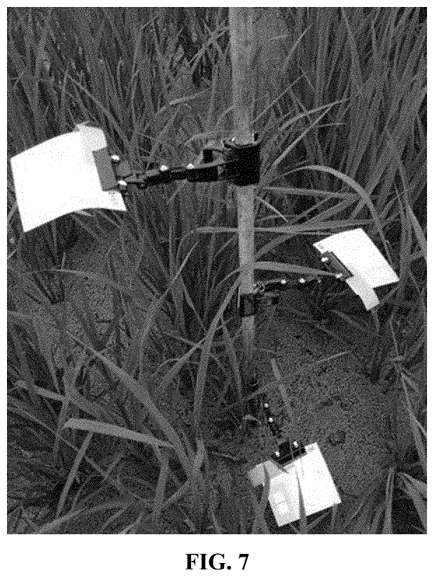

A 1 m steel pipe was inserted into the rice plant, and the detection membranes (prepared by referring to Example 1) were fixed with double-head clips at the height of 10 cm, 40 cm and 70 cm from water surface, which were marked as the lower layer, middle layer and upper layer of rice canopy respectively. In the test, 4 plots were set up, and 10 steel pipes were arranged in each plot (FIG. 7). Spray was applied to the 4 plots of the rice fields by the UAV equipped with Lechler LU 120-015 universal flat fan-shaped spray nozzle (height: 3 m, speed: 5 m/s, and spraying width: 4 m). The spray liquids were spray liquid A (group 1 in Table 1 was selected for probes), spray liquid B (group 5 in Table 1 was selected for probes), spray liquid C (group 1 in Table 1 was selected for probes) and spray liquid D (group 5 in Table 1 was selected for probes), which were marked as treatment 1, treatment 2, treatment 3 and treatment 4 respectively; wherein, spray liquid A contained 0.06 μM transition probe, 40% chlorantraniliprole⋅thiamethoxam water dispersible granules and 1% organic silicon flight prevention adjuvant, and corresponded to the detection membrane carrying immobilized probe 1; spray liquid B contained 0.06 μM transition probe, 40% chlorantraniliprole⋅thiamethoxam water dispersible granules and 1% surfactant flight prevention adjuvant, and corresponded to the detection membrane carrying immobilized probe 2; spray liquid C contained 0.06 μM transition probe, 40% chlorantraniliprole⋅thiamethoxam water dispersible granules and 1% oil flight prevention adjuvant, and corresponded to the detection membrane carrying immobilized probe 1; and spray liquid D contained 0.06 μM transition probe and 40% chlorantraniliprole⋅thiamethoxam water dispersible granules, and corresponded to the detection membrane carrying immobilized probe 2.

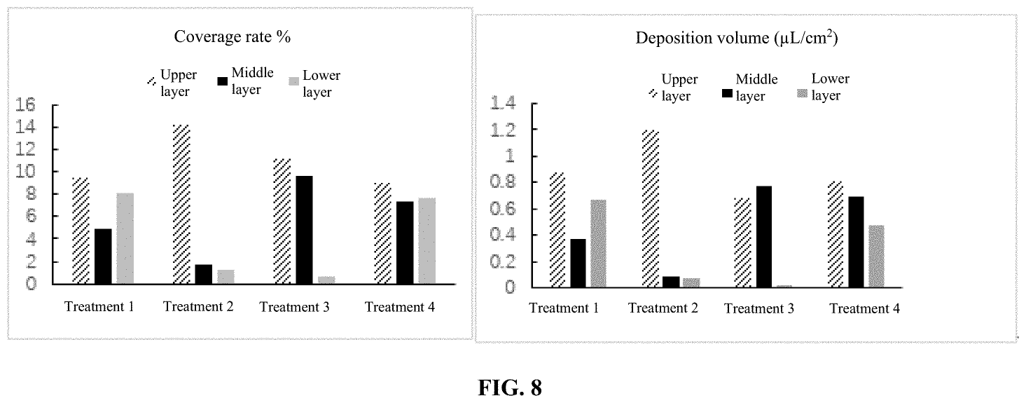

After spraying, the experimental materials were retrieved respectively, and the detection membranes were subjected to chromogenic reactions and drying using corresponding chromogenic probes according to the chromogenic treatment as described in Example 1 to calculate the coverage area and coverage rate of droplets collected by different detection membranes. A digital image was obtained by means such as photographing or scanning, the gray values of unit areas were read by an image processing software (Photoshop and Image J), and a total gray value of a selected area was calculated. Finally, the spray droplet deposition was calculated according to the standard curve. The results are shown in FIG. 8. The results show that this technique can be used for detecting the droplets in rice field experiments under various conditions, and can be combined with the pesticide formulations for direct detection. In addition, the drift and deposition during the spraying process can be well reflected on the amount, coverage rate and deposition volume of the droplets.

### Example 7: Deposit Distribution Test of Cotton Defoliant

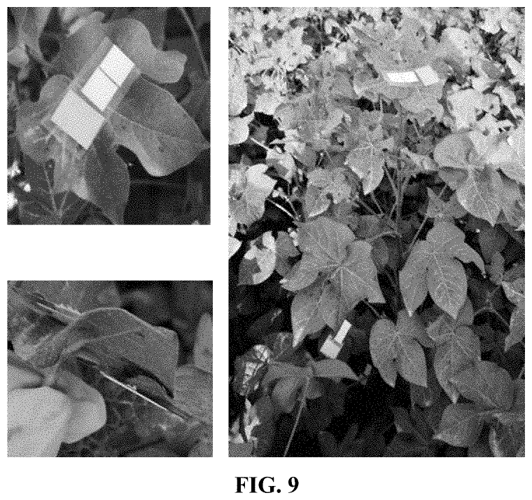

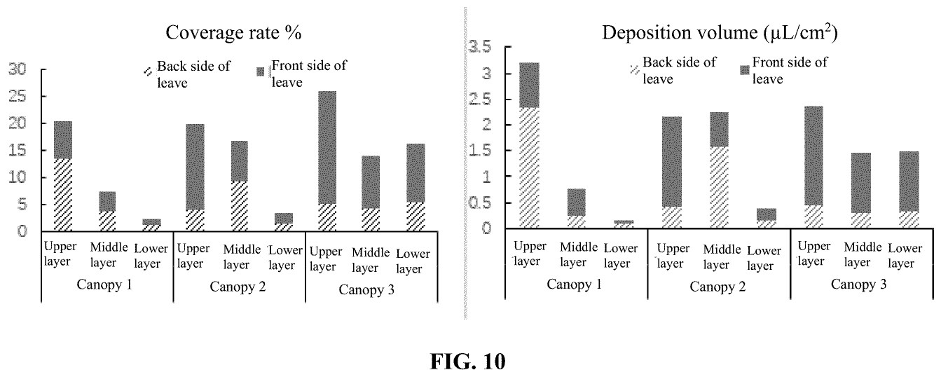

Both front and back sides of detection membranes (prepared by referring to Example 1) were pasted on the selected leaves of the lower, middle and upper layers of cotton plants with a double-sided tape (FIG. 9). In the test, 3 plots were set up, corresponding to cotton test fields with the plant heights of 1.5 m, 1.2 m and 1.0 m respectively. Spray was applied to the 3 plots of the cotton test fields by the UAV equipped with Lechler LU 120-015 universal flat fan-shaped spray nozzle (height: 3 m, speed: 5 m/s, and spraying width: 4 m). The spray liquid contained 0.06 μM transition probe (group 1), 15 mL/L Dropp Ultra and 40 mL/L ethephon. After spraying, the experimental materials were retrieved respectively, and the detection membranes were subjected to chromogenic reactions and drying according to the chromogenic treatment as described in Example 1 to calculate the coverage area and coverage rate of droplets collected by different detection membranes. A digital image was obtained by means such as photographing or scanning, the gray values of unit areas were read by an image processing software (Photoshop and Image J), and a total gray value of a selected area was calculated. Finally, the deposition volume was calculated according to the standard curve. The results are shown in FIG. 10. The results show that this technique can be used for detecting the droplets in cotton defoliant test. As the detection membranes can be pasted on the leave surface due to their light weight, the real leaves conditions can be reflected accurately. In addition, the deposition during the spraying process can be well reflected on the amount, coverage rate and deposition volume of the droplets.

### Example 8: Pesticide Spray Drift Test in an Integrated Rice-Crayfish Farming System

In the canal of shrimp paddy field, foams were used as the sample receiving devices, on which the detection membrane and water sensitive paper were pasted. The canal was divided into sites according to its width. Three receiving devices as a group were placed at each site, and a group was placed every 2 m for 11 groups in total. The detection membranes were prepared by referring to Example 1, and group 1 in Table 1 was selected for immobilized probes on the detection membranes. Spray was applied to the shrimp paddy fields around the canal by the UAV equipped with Lechler LU 120-015 universal flat fan-shaped spray nozzle (height: 3 m, speed: 5 m/s, and spraying width: 4 m). The spray liquid contained 0.06 μM transition probe (group 1 in Table 1) and 10 mL/L thiamethoxam⋅cyalothrin.

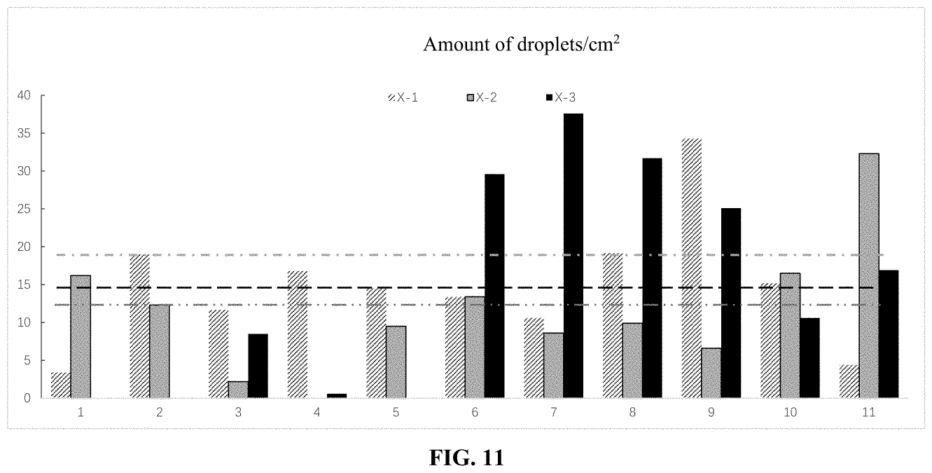

After spraying, the experimental materials were retrieved respectively, and the detection membranes were subjected to chromogenic reactions and drying using chromogenic probes in group 1 of Table 1 according to the chromogenic treatment as described in Example 1 to calculate the coverage area and coverage rate of droplets collected by different detection membranes. A digital image was obtained by means such as photographing or scanning, the gray values of unit areas were read by an image processing software (e.g., Photoshop, Image J and the like), and a total gray value of a selected area was calculated. Finally, the spray droplet drift volume was calculated according to the standard curve. The results are shown in FIG. 11. The results show that this technique can be used for detecting the drift in the integrated rice-crayfish farming system, and can avoid the situation that the water sensitive paper cannot be detected because it turns blue due to moisture. In addition, the droplet drift during the spraying process can be well reflected on the amount, coverage rate and deposition volume of the droplets in this method.

TABLE 2
Detection results of droplet deposition of pesticide in the
integrated rice-crayfish farming system field.

| Num- | Coverage | Droplet | Deposition volume |
|---|---|---|---|
| ber | rate % | amount/cm² | (μL/cm²) |
| 1-1 | 0.10 | 3.4 | 0.003 |
| 1-2 | 0.60 | 16.2 | 0.029 |
| 1-3 | 8.76 | 44.8 | 0.376 |
| 2-1 | 0.69 | 19.0 | 0.029 |
| 2-2 | 1.91 | 12.3 | 0.165 |
| 2-3 | 5.41 | 34.2 | 0.472 |
| 3-1 | 0.32 | 11.7 | 0.010 |
| 3-2 | 0.06 | 2.2 | 0.002 |
| 3-3 | 1.73 | 8.5 | 0.227 |
| 4-1 | 0.62 | 16.8 | 0.022 |
| 4-2 | 6.67 | 23.1 | 0.403 |
| 4-3 | 0.01 | 0.6 | 0.001 |
| 5-1 | 0.89 | 14.5 | 0.040 |
| 5-2 | 2.40 | 9.5 | 0.329 |
| 5-3 | 14.61 | 29.7 | 1.536 |
| 6-1 | 0.47 | 13.4 | 0.016 |
| 6-2 | 0.46 | 13.4 | 0.017 |
| 6-3 | 0.94 | 29.6 | 0.032 |
| 7-1 | 0.29 | 10.6 | 0.010 |
| 7-2 | 2.79 | 8.6 | 0.406 |
| 7-3 | 2.41 | 37.6 | 0.119 |
| 8-1 | 0.76 | 19.2 | 0.027 |
| 8-2 | 1.23 | 9.9 | 0.124 |
| 8-3 | 1.48 | 31.7 | 0.057 |
| 9-1 | 1.19 | 34.3 | 0.046 |
| 9-2 | 0.20 | 6.6 | 0.006 |
| 9-3 | 0.80 | 25.1 | 0.029 |
| 10-1 | 0.33 | 15.2 | 0.009 |
| 10-2 | 0.26 | 16.5 | 0.007 |
| 10-3 | 0.56 | 10.6 | 0.029 |
| 11-1 | 0.13 | 4.4 | 0.004 |
| 11-2 | 1.25 | 32.3 | 0.056 |
| 11-3 | 0.72 | 16.9 | 0.027 |

1. A method for detecting droplet drift or deposition characteristics of spray, comprising the following steps: adding transition probes as tracers to pesticide liquid or liquid fertilizer or other liquid formulations, after spraying, specifically binding the transition probes to the immobilized probes on the detection membranes, wherein the detection membranes are substrates carrying the immobilized probes, and detecting the transition probes on the detection membranes to determine the droplet drift or deposition of spray.

2. The method according to claim 1, wherein the transition probes and the immobilized probes are single-stranded deoxyribonucleic acids with characteristic sequences; the length of the immobilized probe is 12-25 nt; one end of the immobilized probe is amino-modified and covalently binds to an exposed carboxyl of the substrate.

3. The method according to claim 1, wherein the transition probes are not biotinylated.

4. The method according to claim 3, comprising the following steps: after the specific binding of the transition probes to the immobilized probes on the detection membranes, binding the chromogenic probes labeled with biotin to the transition probes through hybridization, performing chromogenic treatment, determining the droplet deposition volume according to the color depth, and determining the coverage rate and the amount of droplets according to the position and the size of chromogenic spots.

5. The method according to claim 4, wherein the complementary pairing region of the chromogenic probe and the transition probe is of 15-40 nt; if the immobilized probe is 5′-labeled, the chromogenic probe is 3′-biotinylated, and if the immobilized probe is 3′-labeled, the chromogenic probe is 5′-biotinylated.

6. The method according to claim 1, wherein the complementary pairing region of the transition probe and the immobilized probe is of 15-25 nt.

7. The method according to claim 1, wherein the detection membrane is prepared according to the following method: acquiring a substrate of a required area, treating the substrate with 0.1-0.3 M HCl, and washing; incubating the substrate in 10-20% EDC solution and washing; incubating the substrate in 0.3-1.0 M NaHCO₃ solution containing 0.025-0.2 μM immobilized probe; and incubating the substrate in NaOH solution, washing and drying; wherein the carboxyl of the substrate is exposed.

8. The method according to claim 7, wherein the detection membrane is prepared according to the following method: acquiring a substrate of a required area, treating the substrate with 0.1 M HCl, and washing; incubating the substrate in 15% EDC for 0.5-1 h and washing; incubating the substrate in 0.5 M NaHCO₃ solution containing 0.03 μM immobilized probe for 10-20 min; and incubating the substrate in 0.05-0.5 M NaOH solution for 5-15 min, washing and drying.

9. A kit for detecting droplet drift or deposition characteristics of spray, comprising detection membranes, transition probes and chromogenic probes, wherein the detection membrane is a substrate carrying the immobilized probes, one end of the immobilized probe is amino-modified, covalently binding to an exposed carboxyl of the substrate, and the substrate is carboxyl-exposed material; the 3′ or 5′ end of the chromogenic probe is labeled with biotin, and the chromogenic probe can specifically bind to the transition probe but cannot specifically bind to the immobilized probe; preferably, the length of the immobilized probe is 12-25 nt, and the length of the transition probe is 24-50 nt.

10. The kit according to claim 9 for detecting droplet drift or deposition characteristics of spray, further comprising a TMB (3,3′,5,5′-tetramethylbenzidine) single-component solution and streptavidin-labeled horseradish peroxidase.
